(19) United States
(12) Patent Application Publication
(10) Pub. No.: US 2013/0203798 A1
(43) Pub. Date: Aug. 8, 2013

(54) DIAGNOSIS AND TREATMENT OF FUNCTIONAL DYSPEPSIA AND IRRITABLE BOWEL SYNDROME

(71) Applicant: EV Med, Lomita, CA (US)

(72) Inventors: John K.S. Chia, Lomita, CA (US); Andrew Chia, Lomita, CA (US)

(73) Assignee: EV MED, Lomita, CA (US)

(21) Appl. No.: 13/758,414

(22) Filed: Feb. 4, 2013

Related U.S. Application Data
(60) Provisional application No. 61/594,848, filed on Feb. 3, 2012.

(51) Int. Cl. G01N 33/569

(52) U.S. Cl. CPC G01N 33/56983
USPC 514/288; 435/5

(57) ABSTRACT

Methods for diagnosing, monitoring, and treating functional dyspepsia and/or irritable bowel syndrome, are disclosed comprising determining the presence of enterovirus in a gastrointestinal biopsy, wherein the presence of enterovirus indicates disease.

### CROSS-REFERENCE TO RELATED APPLICATIONS

The present application claims the benefit under 35 U.S.C. §119(e) of U.S. Provisional Patent Application No. 61/594,848 filed Feb. 3, 2012 which is incorporated by reference herein its entirety.

### FIELD

The present invention relates to methods for diagnosing functional dyspepsia and irritable bowel syndrome. More specifically the invention provides methods for determining the presence or absence of enterovirus protein and/or double stranded RNA in samples taken from a patient's stomach and colon biopsies, wherein the presence of enterovirus indicates disease.

### BACKGROUND

Functional dyspepsia (FD) and irritable bowel syndrome (IBS) are two of the most prevalent and elusive disorders of the gastrointestinal (GI) tract. It is estimated that 12-30% of the general population has some symptoms of FD and IBS. Functional dyspepsia can cause stomach pain, bloating, gas and a feeling of fullness before finishing a meal. Symptoms of IBS include abdominal pain and changes in bowel movement. There is no direct cause for IBS but it is believed that it can be associated with an underlying digestive disease. When severe, a patient with FD or IBS can present with abdominal pain, cyclical vomiting for the former and debilitating pain, bloating and diarrhea alternating with constipation for the latter. These symptoms often require frequent visits to physician's offices and emergency rooms and in some cases can even require hospitalization.

The etiology of FD and IBS remains unknown despite decades of research. The symptoms are believed to be due to abnormal functioning of the muscles and/or nerves supplying the GI tract which extends from the esophagus to the anus. The myenteric plexus is a network of nerves (a plexus of unmyelinated fibers and postganglionic autonomic cell bodies) which lie within the muscular walls/tissue of the esophagus, stomach, and intestines. The function of the local nerve network is further modulated by the central nervous system via the vagus nerve and sympathetic trunk.

Abnormality or injury in the local nerves plexus, vagus nerve, spinal cord or brain can result in significant dysfunction of the GI tract. Abnormal sensory signals coming from GI organs or excessive motor nerve output from the brain can result in symptoms associated with FD and IBS. A disease such as gastritis or colitis can cause permanent changes in the sensitivity of the nerves or processing centers of the GI tract. As a result of this prior inflammation, normal stimuli are perceived as abnormal signals.

It is unclear what diseases might lead to hypersensitivity in people, although bacterial, parasitic and viral diseases of the GI tract are considered triggers. Approximately 20-30% of all patients who develop severe gastrointestinal infections will go on to develop IBS symptoms. Campylobacter jejuni, Salmonella, Shigella and certain strains of Echerichia coli have been associated with the onset of IBS. Results of recent research have identified risk factors for developing IBS following gut infection. These include the severity and duration of the original infection, the female gender, and an onset of pre-existing anxiety and depression. It has been postulated that the offending gut infection results in a subtle but significant inflammation in the gut wall, which can result in the over production of serotonin or other neurotransmitters from certain enterochromatic cells in the gut wall. Mast cell numbers, histamine and tryptase levels were greater in IBS patients. Current treatments for FD and IBS are directed at symptom relief; promoting or inhibiting motility and adjustment of neurotransmitters in the central nervous system.

### SUMMARY

The present disclosure provides methods for diagnosing functional dyspepsia (FD) and/or irritable bowel syndrome (IBS) by determining the presence of enterovirus in a gastric biopsy, wherein the presence of enterovirus indicates disease.

In one embodiment, a method for diagnosing functional dyspepsia and/or irritable bowel syndrome is disclosed, the method comprising obtaining stomach and/or colon biopsies from a subject and assaying for the presence of enterovirus in the sample, whereby the presence of enterovirus indicates the subject suffers from FD or IBS.

In another embodiment, the method includes identifying a component of the enterovirus in the sample wherein the component is a protein, RNA, double-stranded RNA, or a combination thereof.

In yet another embodiment, immunoperoxidase staining is used to detect the presence of enterovirus protein and/or double stranded RNA in the sample.

In still another embodiment, a method for monitoring treatment of functional dyspepsia and/or irritable bowel syndrome is disclosed, the method comprising obtaining one or more biopsies from a subject under treatment and determining the presence of enterovirus in the sample whereby the absence or reduction of enterovirus in a sample obtained after treatment compared to a sample taken at the beginning of treatment indicates that the treatment has been effective.

In another embodiment, treatment or prevention of functional dyspepsia or irritable bowel syndrome comprises administering an effective amount of at least one of an anti-enterovirus composition to a patient in need of such a treatment.

In another embodiment, the composition is oxymatrine, or an oxymatrine-containing composition.

### BRIEF DESCRIPTION OF THE DRAWINGS

FIG. 1 depicts the staining for enteroviral capsid protein (VP1) and dsRNA in stomach biopsies of two patients with functional dyspepsia (FD). FIGS. 1A and B depict tissues of a first patient and demonstrate positive staining for VP1 and dsRNA, respectively (100×magnification). FIGS. 1C and D depict the same for a second patient.

FIG. 2 depicts staining for dsRNA in stomach biopsies with and without RNase III digestion. FIGS. 2A and C are stomach biopsies from FD patients stained for dsRNA without RNAse III digestion (100×magnification). FIGS. 2B and D represent the same specimens with prior RNase III digestion.

FIG. 3A depicts enterorvirus-specific staining of resected muscles from a patient with small bowel obstruction. The staining demonstrates VP1 in the longitudinal and circular muscles, and in the myentric plexus (shown by the arrows). FIG. 3B shows that there was no viral protein detected in another section of the same intestinal segment.

FIG. 4 depicts immunoperoxidase staining of resected colon from a patient with severe IBS and colon inertia. FIG. 4A shows extensive viral protein in the muscle layers (100×magnification). FIG. 4B shows positive viral protein staining of neurons in the myenteric plexus (shown by the arrows) and also in the smooth muscle (400×magnification). FIGS. 4C and D show no staining of the intestinal muscles (100×magnification) and the myenteric plexus (400×magnification), respectively, of a control with a normal colon.

FIG. 5 depicts colon epithelium which stained positive for VP1 by immunoperoxidase staining (100×magnification).

FIG. 6 depicts the effect of oxymatrine on the VP1 and dsRNA in the stomach, before and after treatment.

### DETAILED DESCRIPTION

Although persistent symptoms can follow acute viral gastroenteritis, chronic enteroviral infection of the gastrointestinal (GI) tract has not been demonstrated in irritable bowel syndrome (IBS) patients. In patients with chronic fatigue syndrome (CFS), chronic persistent infection of stomach, small bowel, and colon follow acute infections. Additional information about the presence of enterovirus in gastric tissue of CFS patients can be found in U.S. Pat. No. 7,597,144 which is incorporated by reference herein in its entirety. Similarly, enterovirus infection of the stomach is also found in patients with functional dyspepsia without CFS, along with double stranded RNA in the stomach biopsies of these patients. Persistent enterovirus infection is found in the muscles and neural plexus of small bowel and colon from patients who had acute, recurrent small bowel obstruction or colon infection and severe IBS that progressed to severe colonic inertia.

Disclosed herein are methods of diagnosing, treating, and monitoring the treatment of functional dyspepsia and irritable bowel syndrome. Diagnosis of FD or IBS is determined by identifying the presence of an enterovirus, or a component of an enterovirus, in a sample of stomach or colon biopsy from a patient suspected of suffering from FD or IBS.

Further disclosed herein are methods of treatment of FD or IBS with a composition which is effective for treating enterovirus infections. In certain embodiments, the composition is oxymatrine or an oxymatrine-containing composition.

Immunoperoxidase staining is useful for detecting double stranded RNA and enterovirus protein in stomach and/or colon biopsies for the diagnosis of FD and IBS

In one embodiment, the composition is oxymatrine. Oxymatrine is one of two major alkaloid components found in sophora roots. They are obtained primarily from Sophora japonica (kushen), but also from Sophora subprostrata (shandougen), and from the above ground portion of Sophora alopecuroides. Oxymatrine was first isolated and identified in 1958 and is a unique tetracyclo-quinolizindine alkaloid(s) found only in Sophora species to date. The chemical structure for oxymatrine is depicted in Formula 1. Methods for the isolation and purification of oxymatrine are well known in the art. For additional disclosure of oxymatrine, see U.S. Pat. No. 8,196,293, which is incorporated by reference for all it discloses regarding oxymatrine.

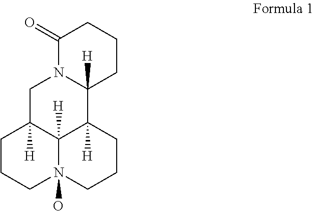

In another embodiment, the composition is an oxymatrine-containing composition. In one embodiment, the oxymatrine-containing composition is a composition including a plant, including the roots, of the genus Sophora, such as Sophora flavescens.

EQUILIBRANT® is an exemplary oxymatrine-containing dietary supplement containing a proprietary blend of vitamins, minerals, and herbal extracts manufactured by Sophora Health LLC and includes vitamin A, vitamin D, calcium, selenium, astragalus root extract, shrubby sophora root extract, olive leaf extract, licorice root extract and shitake mushroom extract.

The compositions described herein may be administered at pharmaceutically effective, anti-infective dosages. Such dosages are normally the minimum dose necessary to achieve the desired therapeutic effect; in the treatment of enterovirus infection, for example, this amount would be roughly that necessary to reduce the symptoms of the enterovirus infection to tolerable levels.

In one embodiment, therapeutic doses of the substantially pure oxymatrine are in the range of 1-1000 mg/day; more preferably in the range of 10 to 500 mg/day. In another example embodiment, the oxymatrine may be present in a composition in a range of about 0.5 mg/kg/day to about 100 mg/kg/day. However, the actual amount of oxymatrine to be administered in any given case will be determined by a physician taking into account the relevant circumstances, such as the severity of the infection, the age and weight of the patient, the patient's general physical condition, and the route of administration.

In one embodiment, therapeutic doses of the substantially pure oxymatrine are administered twice daily at a dose of between about 5 mg and about 500 mg per administration; between about 10 mg and about 450 mg per administration; between about 50 mg and about 375 mg per administration; between about 100 mg and about 250 mg per administration; between about 150 mg and about 225 mg per administration; or between about 175 mg and about 200 mg per administration.

Similar dosage ranges are also suitable as maintenance doses as determined by the attending physician. Essentially, a patient can be treated with progressively smaller doses until an optimum minimal dose is found that suppresses reoccurrences of symptoms. Thereafter, dose frequency can be titrated to determine the minimum dosage schedule necessary to prevent disease reoccurrence at the minimum dose.

In other embodiments, the patient is administered a composition orally in any acceptable form, such as a tablet, liquid, capsule, powder and the like. However, other routes may be desirable or necessary. Such other routes may include, without exception, transdermal, parenteral, subcutaneous, intranasal, intrathecal, intramuscular, intravenous, and intrarectal modes of delivery. Additionally, formulations may be designed to delay release of the active compound over a given period of time, or to carefully control the amount of oxymatrine released at a given time during the course of therapy.

In another embodiment, provided are pharmaceutical compositions including therapeutic doses of the substantially pure oxymatrine in a pharmaceutically acceptable carrier or excipient. The phrase “pharmaceutically acceptable” means the carrier, dilutent or excipient must be compatible with the other ingredients of the formulation and not deleterious to the recipient thereof.

Pharmaceutical compositions can be used in the form of a solid, a solution, an emulsion, a dispersion, a micelle, a liposome, and the like, wherein the resulting composition contains therapeutic doses of the substantially pure oxymatrine as described herein, as an active ingredient, in admixture with an organic or inorganic carrier or excipient suitable for enteral or parenteral applications. Therapeutic doses of the substantially pure oxymatrine may be combined, for example, with the usual non-toxic, pharmaceutically acceptable excipients for tablets, pellets, capsules, suppositories, solutions, emulsions, suspensions, and any other form suitable for use. The excipients which can be used include glucose, lactose, gum acacia, gelatin, mannitol, starch paste, magnesium trisilicate, talc, corn starch, keratin, colloidal silica, potato starch, urea, medium chain length triglycerides, dextrans, and other carriers or excipients suitable for use in manufacturing preparations, in solid, semisolid, or liquid form. In addition, auxiliary, stabilizing, thickening and coloring agents and perfumes may be used. Therapeutic, anti-infective doses of the substantially pure oxymatrine described herein are included in pharmaceutical compositions in an amount sufficient to produce the desired effect upon the infective condition.

Pharmaceutical compositions may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of a sweetening agent such as sucrose, lactose, or saccharin, flavoring agents such as peppermint, oil of wintergreen or cherry, coloring agents and preserving agents in order to provide pharmaceutically elegant and palatable preparations. Tablets containing therapeutic doses of the substantially pure oxymatrine as described herein in admixture with non-toxic pharmaceutically acceptable excipients may also be manufactured by known methods.

The excipients used may be, for example, (1) inert diluents such as calcium carbonate, lactose, calcium phosphate or sodium phosphate; (2) granulating and disintegrating agents such as corn starch, potato starch or alginic acid; (3) binding agents such as gum tragacanth, corn starch, gelatin or acacia, and (4) lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

In some embodiments, formulations for oral use may be in the form of hard gelatin capsules wherein the therapeutic doses of the substantially pure oxymatrine are mixed with an inert solid diluent, for example, calcium carbonate, calcium phosphate or kaolin. They may also be in the form of soft gelatin capsules wherein the invention compounds are mixed with water or an oil medium, for example, peanut oil, liquid paraffin, or olive oil.

The pharmaceutical compositions may also be in the form of a sterile injectable suspension. Suspensions may be formulated according to known methods using suitable dispersing or wetting agents and suspending agents. The sterile injectable preparations may also be sterile injectable solutions or suspensions in a non-toxic parenterally-acceptable diluent or solvent, for example, as a solution in 1,3-butanediol. Sterile, fixed oils are conventionally employed as a solvent or suspending medium. For this purpose, any bland fixed oil may be employed including synthetic mono- or diglycerides, fatty acids, naturally occurring vegetable oils, for example, sesame oil, coconut oil, peanut oil, cottonseed oil, etc., or synthetic fatty vehicles like ethyl oleate or the like. Buffers, preservatives, antioxidants, and the like can be incorporated as required.

Compositions described herein may also be administered in the form of suppositories for rectal administration. These compositions may be prepared by mixing the compounds with a suitable non-irritating excipient, such as cocoa butter, synthetic glyceride esters of polyethylene glycols, which are solid at ordinary temperatures, but liquefy and/or dissolve in the rectal cavity to release the therapeutic dose of the substantially pure oxymatrine.

### EXAMPLES

### Example 1

### Positive Staining for VP1 and dsRNA in Patients with Functional Dyspepsia

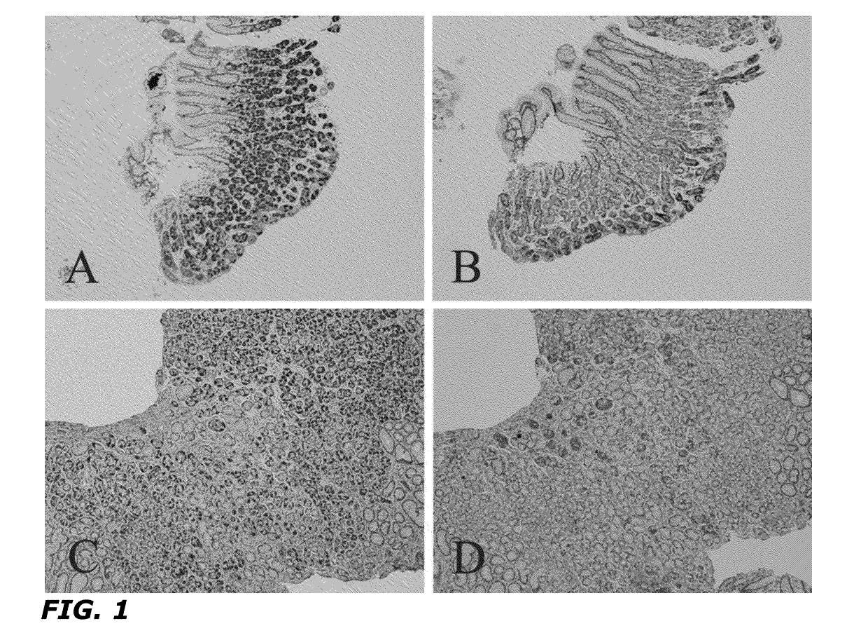

Using monoclonal antibodies against a conserved enterovirus protein epitope (5D8/1, Dako,) and a specific configuration of double-stranded RNA (J2, English & Scientific Consulting, Hungry), VP1 was demonstrated in 84% and 83% of the stomach biopsies tested, and double-stranded RNA (dsRNA) in 64% and 55% of tested FD patients with or without CFS, respectively. Exemplary sections from two patients are depicted in FIG. 1, whereas 9/41 (22%) and 5/41 (12%) of the controls stained positive for VP1 and dsRNA, respectively (p<0.01, X² test). 21/23 (91%) of stomach biopsies previously tested positive for enterovirus RNA by real-time polymerase chain reaction (RT-PCR) or had grown non-cytopathic virus that were positive for dsRNA.

### Example 2

### DsRNA Staining with and without RNAse III Digestion

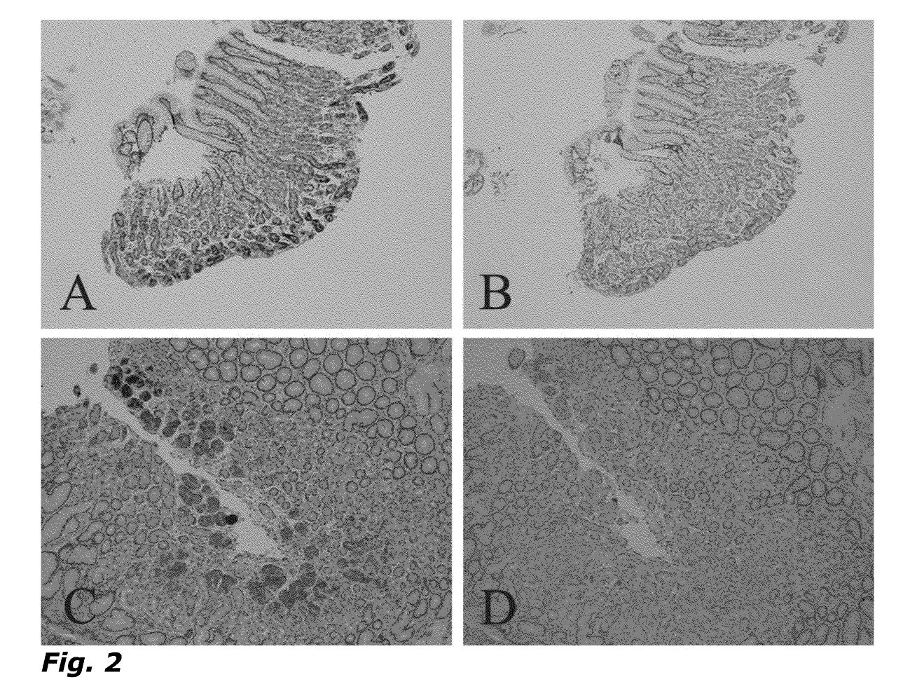

Stomach biopsies from FD patients stained positive for dsRNA without RNAIII digestion (FIG. 2A and B). RNAse III digestion of the stomach biopsies before the immunoperoxidase staining procedure completely abolished the staining (FIG. 2C and D) confirming the RNA sequence is double-stranded. These findings suggest that the persistence of the viral genome is in the form of double stranded RNA, and that expression of enteroviral protein by the infected cells is indicative of active infection without lysis of cells.

### Example 3

### Staining of Resected Muscles in Patient's with Small Bowel Obstruction

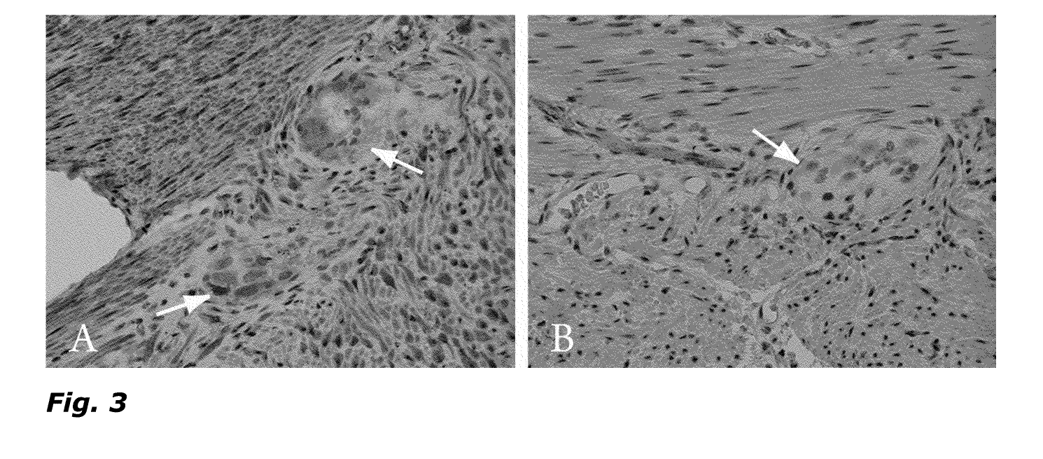

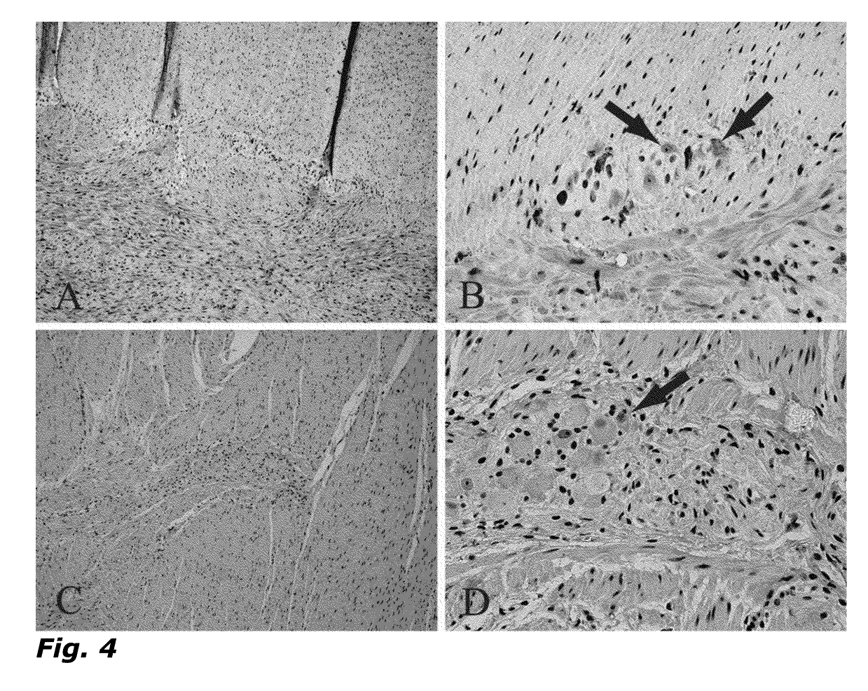

IBS symptoms can be due to involvement of the small bowel and/or colon. Acute enterovirus infection of small intestinal smooth muscles causes intussusception. Several patients who developed acute and recurrent small bowel obstructions (SBO) had infections of longitudinal and circular muscles and myenteric plexus demonstrated in some resected intestingal segments (FIG. 3A) but not in other areas (FIG. 3B). Three patients with acute enterovirus gastroenteritis/colitis, documented by finding positive viral protein of the stomach and colon biopsies, developed severe IBS with progression to colonic inertia leading to total colectomy. A marked reduction of myenteric plexus was noted in the smooth muscles layer, as compared to control colon samples. Immunoperoxidase staining demonstrated enterovirus protein in the smooth muscles and nerve plexus (FIG. 4A and B at 100×, 400×magnification, respectively), and not in normal colon controls (FIG. 4C and D at 100×and 400×magnification, respectively). These findings suggest that chronic infection of nerves and muscles leads to symptoms of IBS, and further destruction of the nerve cells would eventually lead to colonic inertia.

### Example 3

### Positive Staining in Resected Colon

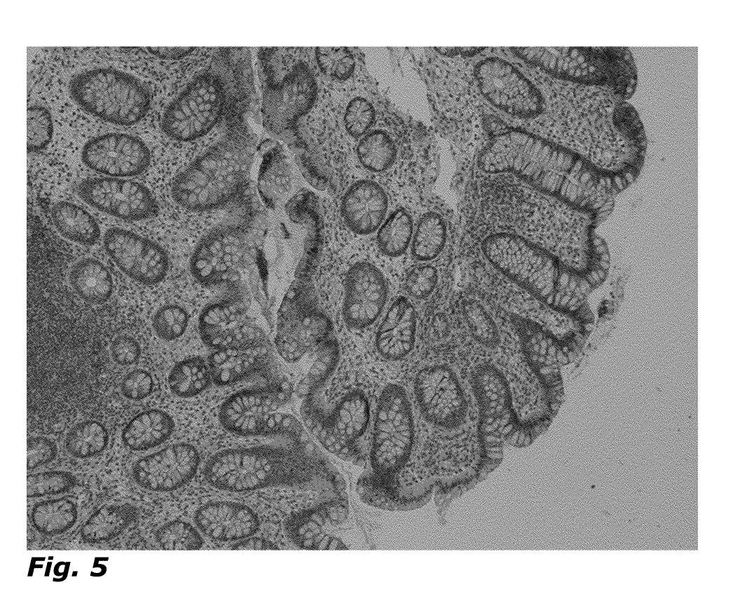

Diagnosis of enterovirus infection in colon smooth muscle is difficult since only superficial mucosa can be sampled at the time of colonoscopy. Five out of five IBS patients who underwent colon resection and had demonstrated positive viral protein in the muscle layers also had evidence of enterovirus in the epithelium (FIG. 5).

### Example 4

### Patients with Symptoms of FD and IBS Stain Positive for VP1

By oral-fecal contamination, enteroviruses reach the stomach before spreading to the small bowel and colon. Thirty-nine out of 68 (57%) patients with symptoms of functional dyspepsia and/or irritable bowel syndrome who had biopsies during colonoscopy, stained positive for EV VP1 in the colon epithelium, as compared to 1 out of 26 (4%) of normal controls (p<0.01, X² test). Of 101 patients with symptoms of functional dyspepsia who tested positive for dsRNA in the stomach biopsy, 87% had mild to severe IBS symptoms and 85% had significant tenderness in lower quadrants of the abdomen. Therefore, the presence of persistent infection in the upper GI tract has significant correlation with lower bowel symptoms.

### Example 5

### Reduction of VP1 and dsRNA with Treatment of Oxymatrine

To further demonstrate the significance of viral infection the gastrointestinal tract as the cause of FD and IBS, reduction of viral protein and dsRNA with treatment should correlate with an improvement of symptoms. Oxymatrine is an alkaloid with antiviral and immune boosting effect. In vitro experiment demonstrated significant inhibition of two out of four enteroviruses when cells were pre-treated with interferon to produce chronic infection (Table 1). 200,000 BGMK-DAF cells were infected with coxsackievirus B3,4 (CVB3,4) or echovirus 6,7 (Echo V6,7), previously titrated to cause complete lysis of cells in 48-72 hours, then treated with 0.18 μg of peg-interferon α-2a in 4 hours and 90 hours. Oxymatrine was added at 162 hours post-infection. Viral RNA, expressed in log₁₀ was quantified by qRT-PCR with a cDNA standard, and reduction of log viral RNA calculated based on no-treatment wells. Duplicate wells were combined for viral RNA determination. Table 1 shows the reduction of viral RNA when treated with several concentrations of oxymatrine.

TABLE 1
Inhibition of enterovirus replications in
chronically infected cells by Oxymatrine

| | BGMK-DAF cells | | | | | | | |
|---|---|---|---|---|---|---|---|---|
| | CVB3 | | CVB4 | | EchoV 6 | | EchoV 7 | |
| | Virus | | | | | | | |
| | | Dec | | Dec | | Dec | | Dec |
| Treatment | Log₁₀ | Log₁₀ | Log₁₀ | Log₁₀ | Log₁₀ | Log₁₀ | Log₁₀ | Log₁₀ |
| No treatment | 6.84 | | 4.20 | | 5.00 | | 5.61 | |
| 100 μM | 1.30 | −5.54 | 0.30 | −3.90 | 5.00 | 0.00 | 5.83 | 0.22 |
| Oxymatrine | | | | | | | | |
| 10 μM | 0.30 | −6.54 | 0.00 | −4.20 | 5.37 | 0.37 | 5.45 | −0.17 |
| Oxymatrine | | | | | | | | |
| 1 μM | 0.60 | −6.24 | 3.66 | −0.55 | 5.00 | 0.00 | 5.33 | −0.28 |
| Oxymatrine | | | | | | | | |

### Example 6

### Treatment of FD with EQUILIBRANT®

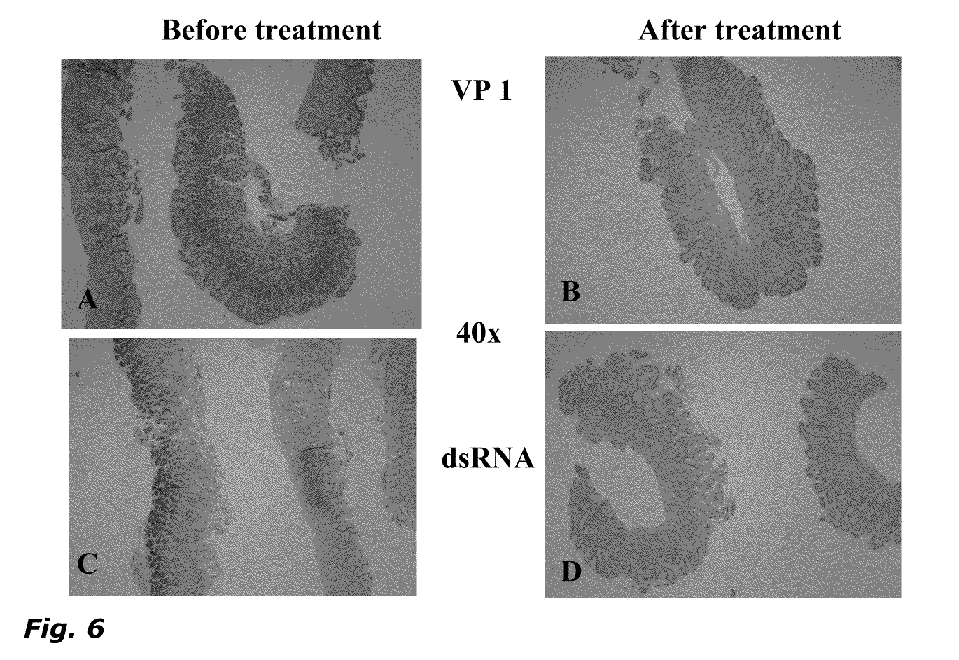

Treatment of patients with CFS and FD with EQUILIBRANT®, an American-made oxymatrine-containing herbal immune booster, resulted in significant improvement of fatigue and gastrointestinal symptoms, and a marked reduction of enteroviral protein and dsRNA in stomach biopsies was demonstrated 2 years after treatment (FIG. 6).

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A method for diagnosing functional dyspepsia and/or irritable bowel syndrome comprising:
assaying for the presence of an enterovirus, or a component thereof, in a stomach or colon biopsy sample from an individual suspected of suffering from functional dyspepsia or irritable bowel disease, whereby the presence of enterovirus indicates the individual suffers from functional dyspepsia and/or irritable bowel syndrome.

3. The method according to claim 1, wherein the component of the enterovirus is a protein, RNA, double-stranded RNA, or a combination thereof.

4. The method according to claim 1, wherein the presence of enterovirus in the sample is determined by immunoperoxidase staining procedures.

5. The method according to claim 1, wherein the sample is a stomach biopsy sample.

6. The method according to claim 1, wherein the sample is a colon biopsy sample.

7. The method according to claim 1, wherein samples are obtained from both stomach and colon and both samples are assayed.

8. A method for monitoring treatment of functional dyspepsia and/or irritable bowel syndrome, comprising:
assaying for the presence of an enterovirus in two or more biopsies from a subject under treatment for functional dyspepsia or irritable bowel syndrome, wherein the biopsy sample is a stomach or colon biopsy sample, whereby the absence or reduction of the enterovirus in a sample obtained after treatment compared to a sample taken before the onset of treatment indicates that the treatment has been effective.

9. The method according to claim 8, wherein the component of the enterovirus is a protein, RNA, double-stranded RNA, or a combination thereof.

10. The method according to claim 8, wherein the presence of enterovirus in the sample is determined by immunoperoxidase staining procedures.

11. The method according to claim 8, wherein the sample is a stomach biopsy sample.

12. The method according to claim 8, wherein the sample is a colon biopsy sample.

13. The method according to claim 8, wherein samples are obtained from both stomach and colon and both samples are assayed.

14. A method for treating functional dyspepsia or irritable bowel syndrome, comprising administering an effective amount of an anti-enterovirus composition to a patient in need of such a treatment.

15. The method according to claim 10, wherein the anti-enterovirus treatment is oxymatrine, or an oxymatrine-containing composition.
